(19) United States
(12) Patent Application Publication
(10) Pub. No.: US 2026/0321409 A1
(43) Pub. Date: Oct. 15, 2026

(54) Method, System, and Computer Program Product for Medical Procedure-Based Inventory Management

(71) Applicant: Mobile Aspects, Inc., Pittsburgh, PA (US)

(72) Inventors: Muhammad R. Rahim, Monroeville, PA (US); Timur P. Sriharto, Monroeville, PA (US); Suneil Mandava, Monroeville, PA (US)

(21) Appl. No.: 19/548,121

(22) Filed: Feb. 24, 2026

Related U.S. Application Data
(60) Provisional application No. 63/765,985, filed on Mar. 3, 2025.

(51) Int. Cl. G16H 40/40; G16H 40/20

(52) U.S. Cl. CPC G16H 40/40; G16H 40/20

(57) ABSTRACT

Described are a method, system, and computer program product for medical procedure-based inventory management. The system includes a processor configured to determine a medical procedure to be performed and receive image data from an imaging device positioned in a room in which the medical procedure is performed. The processor is also configured to input the image data to a machine-learning model trained on images of a plurality of items. The processor is further configured to receive an output from the machine-learning model associated with a classification. The processor is further configured to identify an item based on the output and determine a status of the item. The processor is further configured to update a record stored in a database based on the status of the item.

### CROSS-REFERENCE TO RELATED APPLICATION

This application claims priority to U.S. Provisional Patent App. No. 63/765,985 filed Mar. 3, 2025, titled “Method, System, and Computer Program Product for Medical Procedure-Based Inventory Management”, the entire disclosure of which is incorporated by reference herein.

### BACKGROUND

### 1. Technical Field

This disclosure relates generally to inventory management and, in non-limiting embodiments or aspects, to methods, systems, and computer program products for medical procedure-based inventory management.

### 2. Technical Considerations

Inventory management in hospitals is extremely difficult and complex. Many hospitals, including those with operating theater areas, may have poor knowledge and management of their inventory of items. This may be because items (e.g., supplies, implants, devices) are complex and variable, which may make them difficult to understand due to medical use, naming, and other related reasons. Additionally, items may vary in size, shape, coating, material, traits, and/or the like, for the same category of item, making it difficult to accurately keep an inventory of those items. Moreover, hospital personnel may prioritize their focus on the health and status of a patient (e.g., a patient undergoing a surgical operation) rather than an inventory, which may create opportunities for items to be misplaced, lost, or incorrectly documented. Such errors result in inaccurate inventories, where a true inventory of items does not match the documentation. This creates the danger of a needed item being unavailable when it is expected and needed for an important procedure. It also increases the risk of an item being inadvertently left somewhere it should not be (e.g., inside a patient, in a cleaning room, etc.).

Further to the above, hospitals may be required to document (e.g., for regulatory purposes) various milestones in a medical procedure, including incisions, item implantations, device usage, and/or the like. Documentation may require recording milestone information related to date, time, personnel, patient, location, and/or the like. Existing processes are prone to human error. Necessary information about a procedure may be inadvertently omitted. Furthermore, existing inventory management solutions may require significant infrastructural changes to a hospital, such as attaching physical tracking devices to each individual item.

There is a need in the art for a technical solution to provide enhanced inventory management and cataloging of items in a procedure room, including during and related to patient procedures. There is a further need in the art for such a technical solution to be low impact on hospitals for requiring infrastructural and process changes.

### SUMMARY

Accordingly, provided are improved methods, systems, and computer program products for medical procedure-based inventory management.

According to non-limiting embodiments or aspects, provided is a system for medical procedure-based inventory management. The system includes at least one processor. The at least one processor is configured to determine a medical procedure to be performed on a patient. The at least one processor is also configured to receive image data from at least one imaging device positioned in a room in which the medical procedure is performed. The at least one processor is further configured to input the image data to at least one machine-learning model trained on images of a plurality of items. The at least one processor is further configured to receive an output from the at least one machine-learning model associated with a classification of the image data. The at least one processor is further configured to identify at least one item based on the output. The at least one processor is further configured to determine at least one status of the at least one item based at least partly on at least one of: a location of the at least one item in the room, a movement of the at least one item in the room, a time, or any combination thereof. The at least one processor is further configured to update at least one record stored in at least one database based on the at least one status of the at least one item.

In some non-limiting embodiments or aspects, the at least one processor may also be configured to receive new image data from the at least one imaging device. The at least one processor may also be configured to determine a suggested item at least partly based on the medical procedure and the new image data. The at least one processor may also be configured to generate a notification via at least one computing device positioned in the room, the notification identifying the suggested item and including at least one of: a textual description of the suggested item, an aural description of the suggested item, a visual depiction of the suggested item, or any combination thereof.

In some non-limiting embodiments or aspects, the at least one processor may also be configured to determine a location of the suggested item in the room based on the new image data. The notification generated via the at least one computing device may further identify the location of the suggested item.

In some non-limiting embodiments or aspects, the at least one processor may also be configured to generate an alert via at least one computing device positioned in the room. The alert may be associated with at least one of the following: the at least one status including a status indicative of the at least one item being contaminated, the at least one item not being associated with a set of pre-approved items used for the medical procedure, or any combination thereof.

In some non-limiting embodiments or aspects, the at least one processor may also be configured to determine a contamination status of the at least one status based on the image data. The contamination status may be indicative of the at least one item being contaminated.

In some non-limiting embodiments or aspects, the at least one record may include a patient record. The at least one processor may also be configured to regularly repeat, during the medical procedure, a set of steps. The set of steps may include receiving the image data from the at least one imaging device. The set of steps may also include determining the at least one status of the at least one items. The set of steps may further include updating the patient record based on the at least one status of the at least one item.

In some non-limiting embodiments or aspects, the at least one processor may also be configured to receive new image data from the at least one imaging device, and determine at least one personnel in the room based on the new image data. When updating the at least one record, the at least one processor may be configured to associate the at least one personnel with the at least one item in the at least one record.

In some non-limiting embodiments or aspects, the at least one processor may be further configured to determine a use of the at least one item based on the image data. The use of the at least one item may be associated with at least one step of a plurality of steps of the medical procedure. When updating the at least one record, the at least one processor may be configured to log the use of the at least one item based on the medical procedure and a time when the use was completed.

In some non-limiting embodiments or aspects, the at least one processor may also be configured to filter the image data in real-time to censor at least one body part of the patient.

In some non-limiting embodiments or aspects, the at least one processor may be configured to determine at least one of: the location of the at least one item in the room, the movement of the at least one item in the room, the time, or any combination thereof.

According to some non-limiting embodiments or aspects, provided is a computer-implemented method for medical procedure-based inventory management. The method includes determining, with at least one processor, a medical procedure to be performed on a patient. The method also includes receiving, with at least one processor, image data from at least one imaging device positioned in a room in which the medical procedure is performed. The method further includes inputting, with at least one processor, the image data to at least one machine-learning model trained on images of a plurality of items. The method further includes receiving, with at least one processor, an output from the at least one machine-learning model associated with a classification of the image data. The method further includes identifying, with at least one processor, at least one item based on the output. The method further includes determining, with at least one processor, at least one status of the at least one item based at least partly on at least one of: a location of the at least one item in the room, a movement of the at least one item in the room, a time, or any combination thereof. The method further includes updating, with at least one processor, at least one record stored in at least one database based on the at least one status of the at least one item.

In some non-limiting embodiments or aspects, the method may further include receiving, with at least one processor, new image data from the at least one imaging device. The method may further include determining, with at least one processor, a suggested item at least partly based on the medical procedure and the new image data. The method may further include generating, with at least one processor, a notification via at least one computing device positioned in the room, the notification identifying the suggested item and including at least one of: a textual description of the suggested item, an aural description of the suggested item, a visual depiction of the suggested item, or any combination thereof.

In some non-limiting embodiments or aspects, the method may further include generating, with at least one processor, an alert via at least one computing device positioned in the room. The alert may be associated with at least one of the following: the at least one status including a status indicative of the at least one item being contaminated, the at least one item not being associated with a set of pre-approved items used for the medical procedure, or any combination thereof.

In some non-limiting embodiments or aspects, the at least one record may include a patient record. The method may further include regularly repeating, during the medical procedure, a set of steps. The set of steps may include receiving, with at least one processor, the image data from the at least one imaging device. The set of steps may also include determining, with at least one processor, the at least one status of the at least one item. The set of steps may further include updating, with at least one processor, the patient record based on the at least one status of the at least one item.

In some non-limiting embodiments or aspects, the method may further include receiving, with at least one processor, new image data from the at least one imaging device. The method may further include determining, with at least one processor, at least one personnel in the room based on the new image data. Updating the at least one record may further include associating the at least one personnel with the at least one item in the at least one record.

According to some non-limiting embodiments or aspects, provided is a computer program product for medical procedure-based inventory management. The computer program product includes at least one non-transitory computer-readable medium including program instructions that, when executed by at least one processor, cause the at least one processor to determine a medical procedure to be performed on a patient. The program instructions also cause the at least one processor to receive image data from at least one imaging device positioned in a room in which the medical procedure is performed. The program instructions further cause the at least one processor to input the image data to at least one machine-learning model trained on images of a plurality of items. The program instructions further cause the at least one processor to receive an output from the at least one machine-learning model associated with a classification of the image data. The program instructions further cause the at least one processor to identify at least one item based on the output. The program instructions further cause the at least one processor to determine at least one status of the at least one item based at least partly on at least one of: a location of the at least one item in the room, a movement of the at least one item in the room, a time, or any combination thereof. The program instructions further cause the at least one processor to update at least one record stored in at least one database based on the at least one status of the at least one item.

In some non-limiting embodiments or aspects, the program instructions may also cause the at least one processor to receive new image data from the at least one imaging device. The program instructions may further cause the at least one processor to determine a suggested item at least partly based on the medical procedure and the new image data. The program instructions may further cause the at least one processor to generate a notification via at least one computing device positioned in the room, the notification identifying the suggested item and including at least one of: a textual description of the suggested item, an aural description of the suggested item, a visual depiction of the suggested item, or any combination thereof.

In some non-limiting embodiments or aspects, the program instructions may also cause the at least one processor to generate an alert via at least one computing device positioned in the room. The alert may be associated with at least one of the following: the at least one status including a status indicative of the at least one item being contaminated, the at least one item not being associated with a set of pre-approved items used for the medical procedure, or any combination thereof.

In some non-limiting embodiments or aspects, the at least one record may include a patient record. The program instructions may also cause the at least one processor to regularly repeat, during the medical procedure, a set of steps. The set of steps may include receiving the image data from the at least one imaging device. The set of steps may also include determining the at least one status of the at least one item. The set of steps may further include updating the patient record based on the at least one status of the at least one item.

In some non-limiting embodiments or aspects, the program instructions may also cause the at least one processor to receive new image data from the at least one imaging device. The program instructions may also cause the at least one processor to determine at least one personnel in the room based on the new image data. The program instructions that cause the at least one processor to update the at least one record may cause the at least one processor to associate the at least one personnel with the at least one item in the at least one record.

Further non-limiting embodiments or aspects are set forth in the following numbered clauses:

Clause 1: A system comprising: at least one processor configured to: determine a medical procedure to be performed on a patient; receive image data from at least one imaging device positioned in a room in which the medical procedure is performed; input the image data to at least one machine-learning model trained on images of a plurality of items; receive an output from the at least one machine-learning model associated with a classification of the image data; identify at least one item based on the output; determine at least one status of the at least one item based at least partly on at least one of: a location of the at least one item in the room, a movement of the at least one item in the room, a time, or any combination thereof; and update at least one record stored in at least one database based on the at least one status of the at least one item.

Clause 2: The system of clause 1, wherein the at least one processor is also configured to: receive new image data from the at least one imaging device; determine a suggested item at least partly based on the medical procedure and the new image data; and generate a notification via at least one computing device positioned in the room, the notification identifying the suggested item and comprising at least one of: a textual description of the suggested item, an aural description of the suggested item, a visual depiction of the suggested item, or any combination thereof.

Clause 3: The system of clause 1 or clause 2, wherein the at least one processor is also configured to determine a location of the suggested item in the room based on the new image data, wherein the notification generated via the at least one computing device further identifies the location of the suggested item.

Clause 4: The system of any of clauses 1-3, wherein the at least one processor is also configured to generate an alert via at least one computing device positioned in the room, the alert associated with at least one of the following: the at least one status comprising a status indicative of the at least one item being contaminated, the at least one item not being associated with a set of pre-approved items used for the medical procedure, or any combination thereof.

Clause 5: The system of any of clauses 1-4, wherein the at least one processor is also configured to determine a contamination status of the at least one status based on the image data, the contamination status indicative of the at least one item being contaminated.

Clause 6: The system of any of clauses 1-5, wherein the at least one record comprises a patient record, and wherein the at least one processor is also configured to regularly repeat, during the medical procedure: receiving the image data from the at least one imaging device; determining the at least one status of the at least one item; and updating the patient record based on the at least one status of the at least one item.

Clause 7: The system of any of clauses 1-6, wherein the at least one processor is also configured to: receive new image data from the at least one imaging device; and determine at least one personnel in the room based on the new image data; and wherein, when updating the at least one record, the at least one processor is configured to: associate the at least one personnel with the at least one item in the at least one record.

Clause 8: The system of any of clauses 1-7, wherein the at least one processor is further configured to determine a use of the at least one item based on the image data, the use of the at least one item associated with at least one step of a plurality of steps of the medical procedure; and wherein, when updating the at least one record, the at least one processor is configured to: log the use of the at least one item based on the medical procedure and a time when the use was completed.

Clause 9: The system of any of clauses 1-8, wherein the at least one processor is also configured to filter the image data in real-time to censor at least one body part of the patient.

Clause 10: The system of any of clauses 1-9, wherein the at least one processor is configured to determine at least one of: the location of the at least one item in the room, the movement of the at least one item in the room, the time, or any combination thereof.

Clause 11: A computer-implemented method comprising: determining, with at least one processor, a medical procedure to be performed on a patient; receiving, with at least one processor, image data from at least one imaging device positioned in a room in which the medical procedure is performed; inputting, with at least one processor, the image data to at least one machine-learning model trained on images of a plurality of items; receiving, with at least one processor, an output from the at least one machine-learning model associated with a classification of the image data; identifying, with at least one processor, at least one item based on the output; determining, with at least one processor, at least one status of the at least one item based at least partly on at least one of: a location of the at least one item in the room, a movement of the at least one item in the room, a time, or any combination thereof; and updating, with at least one processor, at least one record stored in at least one database based on the at least one status of the at least one item.

Clause 12: The computer-implemented method of clause 11, further comprising: receiving, with at least one processor, new image data from the at least one imaging device; determining, with at least one processor, a suggested item at least partly based on the medical procedure and the new image data; and generating, with at least one processor, a notification via at least one computing device positioned in the room, the notification identifying the suggested item and comprising at least one of: a textual description of the suggested item, an aural description of the suggested item, a visual depiction of the suggested item, or any combination thereof.

Clause 13: The computer-implemented method of clause 11 or clause 12, further comprising generating, with at least one processor, an alert via at least one computing device positioned in the room, the alert associated with at least one of the following: the at least one status comprising a status indicative of the at least one item being contaminated, the at least one item not being associated with a set of pre-approved items used for the medical procedure, or any combination thereof.

Clause 14: The computer-implemented method of any of clauses 11-13, wherein the at least one record comprises a patient record, the method further comprising regularly repeating, during the medical procedure: receiving, with at least one processor, the image data from the at least one imaging device; determining, with at least one processor, the at least one status of the at least one item; and updating, with at least one processor, the patient record based on the at least one status of the at least one item.

Clause 15: The computer-implemented method of any of clauses 11-14, further comprising: receiving, with at least one processor, new image data from the at least one imaging device; and determining, with at least one processor, at least one personnel in the room based on the new image data; wherein updating the at least one record further comprises: associating the at least one personnel with the at least one item in the at least one record.

Clause 16: A computer program product comprising at least one non-transitory computer-readable medium comprising program instructions that, when executed by at least one processor, cause the at least one processor to: determine a medical procedure to be performed on a patient; receive image data from at least one imaging device positioned in a room in which the medical procedure is performed; input the image data to at least one machine-learning model trained on images of a plurality of items; receive an output from the at least one machine-learning model associated with a classification of the image data; identify at least one item based on the output; determine at least one status of the at least one item based at least partly on at least one of: a location of the at least one item in the room, a movement of the at least one item in the room, a time, or any combination thereof; and update at least one record stored in at least one database based on the at least one status of the at least one item.

Clause 17: The computer program product of clause 16, wherein the program instructions also cause the at least one processor to: receive new image data from the at least one imaging device; determine a suggested item at least partly based on the medical procedure and the new image data; and generate a notification via at least one computing device positioned in the room, the notification identifying the suggested item and comprising at least one of: a textual description of the suggested item, an aural description of the suggested item, a visual depiction of the suggested item, or any combination thereof.

Clause 18: The computer program product of clause 16 or clause 17, wherein the program instructions also cause the at least one processor to generate an alert via at least one computing device positioned in the room, the alert associated with at least one of the following: the at least one status comprising a status indicative of the at least one item being contaminated, the at least one item not being associated with a set of pre-approved items used for the medical procedure, or any combination thereof.

Clause 19: The computer program product of any of clauses 16-18, wherein the at least one record comprises a patient record, and wherein the program instructions also cause the at least one processor to regularly repeat, during the medical procedure: receiving the image data from the at least one imaging device; determining the at least one status of the at least one item; and updating the patient record based on the at least one status of the at least one item.

Clause 20: The computer program product of any of clauses 16-19, wherein the program instructions also cause the at least one processor to: receive new image data from the at least one imaging device; and determine at least one personnel in the room based on the new image data; and wherein the program instructions that cause the at least one processor to update the at least one record cause the at least one processor to: associate the at least one personnel with the at least one item in the at least one record.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1 is a schematic diagram of a system for medical procedure-based inventory management, according to some non-limiting embodiments or aspects;

FIG. 2 is a schematic diagram of example components of one or more devices of FIG. 1, according to some non-limiting embodiments or aspects;

FIG. 3 is a flow diagram of a method for medical procedure-based inventory management, according to some non-limiting embodiments or aspects;

FIG. 4 is a flow diagram of a method for medical procedure-based inventory management, according to some non-limiting embodiments or aspects;

FIG. 5 is an illustration of a procedure room configured for a method for medical procedure-based inventory management, according to some non-limiting embodiments or aspects; and

FIG. 6 is an illustration of a top surface of a medical instrument stand for use with a method for medical procedure-based inventory management, according to some non-limiting embodiments or aspects.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the embodiments as they are oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects of the disclosed subject matter. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the term “server” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, desktop computers, mobile devices, etc.) directly or indirectly communicating in the network environment may constitute a “system.”

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

As used herein, “real-time” refers to the capability of a system or process to receive, process, and respond to inputs or events within a timeframe that is imperceptible to the user or in accordance with the constraints of the application domain. In the context of machine learning, real-time denotes the ability to perform data processing, analysis, and decision-making instantly or within milliseconds, ensuring that the output or action is delivered virtually immediately after the input is received, thus enabling timely and responsive operation.

The methods, systems, and computer program products described herein provide numerous technical advantages in systems for inventory management. For example, the described systems and methods allow for precise identification and analysis of items within a room for a medical procedure, by inputting image data from an imaging device positioned in the room to a machine-learning model trained to classify such items. Machine-learning models may be trained to not only classify the general category of item (e.g., endoscope, forceps, etc.), but also to specifically classify items within a general category (e.g., bronchoscope, colonoscope, gastroscope, etc.; also dressing forceps, hemostatic forceps, tissue forceps, etc.), providing near real-time classification and identification of items. This may provide added layers of verification to manual inspection and/or relieve medical personnel from executing constant supervision of items in the room. Furthermore, by using machine-learning analysis of image data, statuses of items may be assessed (e.g., contamination status, use status, position status, movement status, cleaning status, etc.), which allow for enhanced processes in the medical procedure space, including, but not limited to: suggesting an item to medical personnel, which item should be used in the medical procedure, alerting medical personnel when an item has become contaminated, alerting medical personnel when an un-approved item is not associated with the medical procedure, identifying the location of an item in the room, and/or the like. Each of these examples represent applied system feedback that lowers the risk for patient infection, reduces opportunities for medical malpractice, and shortens medical procedure time.

Described systems and methods also provide the technical benefits of more sophisticated patient record tracking and logging. For example, each item used in a medical procedure may be logged and associated with a stored record (e.g., a patient record), including a variety of data points including, but not limited to: time of use, personnel using the item, step of medical procedure used, and/or the like. Such tracking features help prevent items from being left on or in a patient, promotes medical personnel accountability, and lowers opportunities for misplacement of items in or around procedure rooms. Such tracking features also reduce the number of attention-based tasks that medical personnel need to perform in a procedure room, which reduces the number of potential non-sterile contact points, reduces opportunities for human error, and maximizes the attention of personnel on performance of the medical procedure itself.

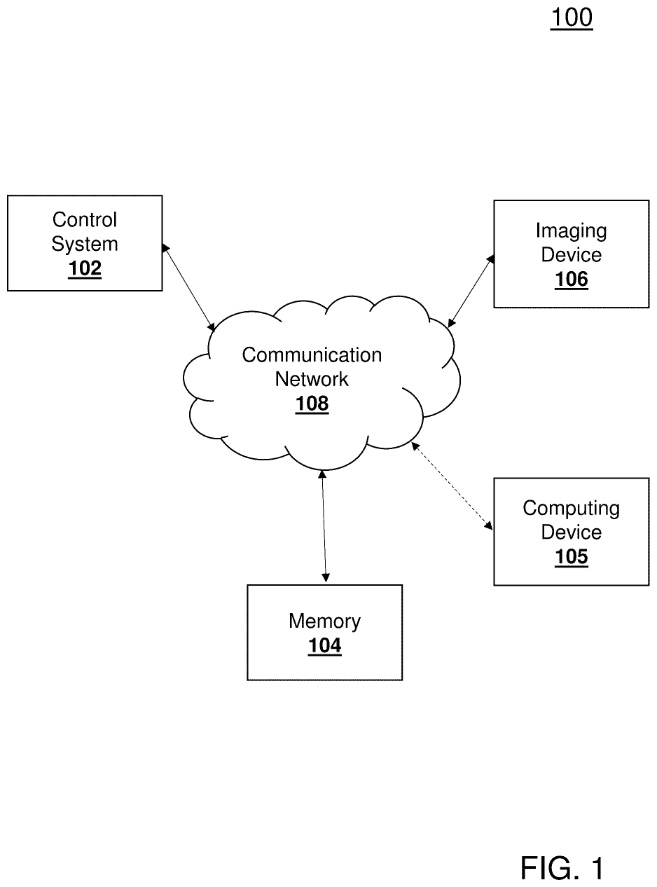

Referring now to FIG. 1, shown is a schematic diagram of a system 100 for medical procedure-based inventory management, according to some non-limiting embodiments or aspects. As shown in FIG. 1, system 100 may include control system 102, memory 104, computing device 105, imaging device 106, and communication network 108. Control system 102, memory 104, computing device 105, and imaging device 106 may interconnect (e.g., establish a connection to communicate) via wired connections, wireless connections, or a combination of wired and wireless connections.

Control system 102 may include one or more computing devices configured to communicate with memory 104, computing device 105, and/or imaging device 106 at least partly over communication network 108. Control system 102 may be configured to receive training data (e.g., training images, such as of items, personnel, furniture, fixtures, etc.) to train one or more image-classification machine-learning models, input new data (e.g., image data generated by imaging device 106) to image-classification machine-learning models, and receive output (e.g., classifications) from image-classification machine-learning models based on the input of new data. Control system 102 may also be configured to receive image data from imaging device 106, detect and identify items and people within a room, track said items and people, and document (e.g., update stored records associated with) the items and people over time. Control system 102 may include or be in communication with memory 104. Control system 102 may further be communicatively connected to an external notification system (e.g., a server associated with one or more manufacturers, regulatory bodies, government entities, etc.), to receive information about items that are being tracked (e.g., recall notices). In some non-limiting embodiments or aspects, control system 102 may be deployed on a server cluster, e.g., a hospital server system, that may be on-site or off-site relative to a given procedure room.

Memory 104 may include one or more computing devices configured to communicate with control system 102, computing device 105, and/or imaging device 106 at least partly over communication network 108. Memory 104 may be configured to data in one or more non-transitory computer readable storage media. For example, memory 104 may store image data generated by imaging device 106. Memory 104 may be configured to store at least one record (e.g., a data entry having multiple fields) associated with at least one item of an inventory of a hospital, at least one patient, at least one personnel, etc. Records stored in memory 104 may be updated (e.g., generated, added to, deleted from, modified, etc.) by control system 102. A record associated with an item and stored in memory 104 may include, but is not limited to, an item identifier, a location of the item, a time of last location update, a patient identifier, a patient procedure identifier (e.g., of a surgical operation), a personnel identifier (e.g., of a clinician, such as a doctor, nurse, or other medical staff), expiration date of the item, cost/value of the item (e.g., in dollars), a status of the item (e.g., a contamination status, a use status, a cleaning status, etc.) and/or the like. Old records of items may be persisted in memory 104, and updates to a record may be maintained with a record (e.g., as an appended table). Memory 104 may communicate with and/or be included in control system 102. Memory 104 may be further configured to store data of one or more image-classification machine learning models. Memory 104 may further be configured to store data of expiration dates or shelf-lives of items.

Computing device 105 may include one or more processors that are configured to communicate with control system 102, memory 104, and/or imaging device 106 at least partly over communication network 108. Computing device 105 may include one or more user interfaces for presenting data to a user and receiving input from a user. For example, computing device 105 may receive and at least partly display communications from control system 102 about one or more items in an inventory or room, such as a communication containing an inventory report, a communication notifying the user about a recall notice, a communication notifying the user about an expired item that needs to be replaced, a communication notifying personnel of a suggested item for use in a medical procedure, a communication alerting personnel of a contaminated item, a communication alerting personnel of an item that is not associated with a set of pre-approved items used for a medical procedure, and/or the like. In some non-limiting embodiments or aspects, computing device 105 may include imaging device 106.

Imaging device 106 may include one or more processors that are configured to communicate with control system 102, computing device 105, and/or memory 104 at least partly over communication network 108. Imaging device 106 may include at least one camera for generating image data (e.g., data associated with one or more images captured by at least one camera). The image data generated by imaging device 106 may be static (e.g., a picture) or dynamic (e.g., video). The image data may include a stream of images (e.g., a number of pictures taken over time, a video feed, etc.). In some non-limiting embodiments or aspects, imaging device 106 may be a mobile device. In some non-limiting embodiments or aspects, imaging device 106 may be a mounted camera on a wall or ceiling of a room of a hospital. In some non-limiting embodiments or aspects, imaging device 106 may be a mounted camera on a fixed or mobile computer station. System 100 may include one or more of imaging devices 106 distributed throughout one or more rooms of a hospital. In some non-limiting embodiments or aspects, imaging device 106 may be associated with or included in a same device as computing device 105.

Communication network 108 may include one or more wired and/or wireless networks over which the systems and devices of system 100 may communicate. For example, communication network 108 may include a cellular network (e.g., a long-term evolution (LTE®) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The number and arrangement of systems and devices shown in FIG. 1 are provided as an example. There may be additional systems and/or devices, fewer systems and/or devices, different systems and/or devices, or differently arranged systems and/or devices than those shown in FIG. 1. Furthermore, two or more systems or devices shown in FIG. 1 may be implemented within a single system or device, or a single system or device shown in FIG. 1 may be implemented as multiple, distributed systems or devices. Additionally, or alternatively, a set of systems (e.g., one or more systems) or a set of devices (e.g., one or more devices) of system 100 may perform one or more functions described as being performed by another set of systems or another set of devices of system 100.

In some non-limiting embodiments or aspects, control system 102 may be configured to determine a medical procedure (e.g., a series of one or more steps carried out by medical personnel on a patient, such as an in-patient operation, an out-patient operation, a configuration of a medical device on the patient, a removal of a medical device from a patient, etc.) to be performed on a patient (e.g., a human admitted to a hospital, in a care center, in a triage unit, etc.). In some non-limiting embodiments or aspects, control system 102 may determine the medical procedure based on a lookup of a patient record stored in memory 104 that identifies the medical procedure to be performed at a specific location, time, etc. Control system 102 may also determine the medical procedure to be performed based on feedback from personnel (e.g., front desk staff, nurse, scheduler, etc.) interacting with a user interface of computing device 105 (e.g., selection and/or specification of a medical procedure, such as from a list of medical procedures). Upon determination of the medical procedure to be performed, control system 102 may retrieve information about the medical procedure (e.g., from memory 104), such as by receiving data stored in association with a patient identifier and/or medical procedure identifier. Control system 102 may determine, for example, a set of pre-approved items that may be used for the medical procedure, a set of steps to be performed for the medical procedure, cleaning instructions for items to be used in the medical procedure, item records for items in an inventory that match the set of pre-approved items, and/or the like.

In some non-limiting embodiments or aspects, control system 102 may be configured to receive image data from at least one imaging device 106 positioned in a room (e.g., a surgical theater, an operation room, an evaluation room, a triage room, etc.) in which the medical procedure is performed (e.g., has been performed, is being performed, will be performed, etc.). Imaging devices 106 may be fixed in the room (e.g., on a wall, on the ceiling, on furniture, on a computer station, etc.), and/or may be mobile in the room (e.g., in a mobile device, on a mobile cart, on a mobile computer station, etc.). Each of the one or more imaging devices 106 that are used to obtain image data related to a medical procedure may communicate image data to control system 102 (e.g., in real-time as the medical procedure is taking place). The image data may be associated with visual information pertaining to one or more items in the room, one or more patients in the room, one or more personnel (e.g., doctors, surgeons, technicians, specialists, nurses, janitors, staff members, etc.) in the room, one or more objects in the room, one or more surfaces in the room, and/or the like. An “item” may include any device, tool, instrument, container, component, or substance utilized, directly or indirectly, in the performance of a medical procedure. An “object” may include any item, as well as other elements in the room, such as furniture, beds, computing devices, carts, tables, lights, and/or the like, and may include humans (e.g., to discern between non-human objects and human-type objects, which may require further classification to determine the identity of the human). Control system 102 may be configured to detect and identify one or more items, objects, personnel, patients, and/or the like.

In some non-limiting embodiments or aspects, control system 102 may input the image data received from at least one imaging device 106 to at least one machine-learning model (e.g., a convolutional neural network (CNN) model, a support vector machine (SVM) model, a random forest model, and/or the like) that is trained on a plurality of images, such as images of items, objects, people, and/or the like. For example, the plurality of images used to train the one or more machine-learning models may include a corpus of images in various formats (e.g., Joint Photographic Experts Group (JPEG) format, Portable Network Graphic (PNG) format, etc.) that may vary in resolution and size. Each training image may be labeled with a ground truth category or class that the image belongs to (e.g., a description/identity of an item, object, personnel, patient, etc.). Training image data may be preprocessed before use for training, such as, but not limited to, normalization (e.g., of pixel values), resizing, augmentation (e.g., rotating, flipping, scaling, etc.). Once trained, a machine-learning model may be configured to classify new images input to the model (e.g., input to an input layer of the machine learning model) to predict the class (e.g., category) to which the new input image most likely belongs. A single machine-learning model may be trained to classify items, objects, personnel, and/or patients, or multiple machine-learning models may be trained on a narrower set of input (e.g., only items, only objects, only humans, only non-human objects, etc.).

In some non-limiting embodiments or aspects, control system 102 may receive an output from the at least one machine-learning model associated with a classification of the image data. In some non-limiting embodiments or aspects, the output of the trained machine-learning model may include a vector of probabilities, one for each possible classification of the image (e.g., each possible classification of an item, object, personnel, patient, etc.). For example, the raw predictions produced by the model's last layer (e.g., output layer) may include logits that may be converted to a probability distribution using a softmax function. Control system 102 may, therefore, determine a classification of a visual artifact in an image based on a class with the highest probability. In some non-limiting embodiments or aspects, a model's final output may be one or more class labels that have the highest probability. Additionally, or alternatively, the output of a model may be paired with an uncertainty or rejection score related to the confidence of the classification.

In some non-limiting embodiments or aspects, control system 102 may identify at least one item, object, personnel, and/or patient based on the output of the at least one machine-learning model (e.g., based on a single or highest-probability classification associated with the output). For example, control system 102 may determine an identity of at least one item in the room based on the output of the machine-learning model trained on a corpus of item identifications. By way of further example, if the relevant model predicts that an item in the room is a “10.0 mm laparoscopic device” at a probability of 85%, and a “5.0 mm laparoscopic device” at a probability of 10%, control system 102 may determine the identity of the item as the “10.00 mm laparoscopic device”. It will be appreciated that the machine-learning model may be trained to output classifications at a chosen level of granularity that is suitable for the application. For example, and for the purposes of illustration only, a model may classify, and control system 102 may identify, an item as broadly as “human” or “tool” or “forceps”, or more specifically as “patient” or “dressing forceps” or “tissue forceps”, or more specifically as “John D. Smith” or “splinter forceps—straight 11.5 cm” or “splinter forceps—product code SD13-1400”, including specifying to the individuality of an item identity (e.g., “patient ID 08749338”, “splinter forceps #158437”, and/or the like).

In some non-limiting embodiments or aspects, control system 102 may determine at least one status of at least one item, object, personnel, and/or patient based at least partly on image data received from at least one imaging device 106. Additionally, or alternatively, control system 102 may determine at least one status of at least one item, object, personnel, and/or patient based at least partly on a location of the subject in the room, a movement of the subject in the room, a time of observation in the room, and/or the like (e.g., which may be ascertained at least partly from image data). For example, for items, statuses may include information related to, but not limited to, a contamination status (e.g., an indicator of level or binary related to the item being contaminated, such as with soiling, bacteria, human contact, expiry, etc.), use status (e.g., being held by a personnel, being placed on or in a patient, being cleaned, etc.), position status (e.g., room, location, associated storage device, associated personnel, associated patient, etc.), movement status (e.g., stationary, moving, directional vectors, state of transfer, etc.), cleaning status (e.g., time since last cleaning, need for cleaning, etc.), and/or the like. By way of further example, statuses for objects, items, personnel, and patients may be related to location, disposition, state of being, involvement in a medical procedure, and/or the like. Information such as location, movement, and/or time may be determined from image data and/or external data provided by other devices, such as tracking devices (e.g., radio frequency identification (RFID) tags, accelerometers, etc.), system/server clocks, stored records, etc. Control system 102 may determine the location and movement of items, objects, personnel, and patients in the room based on the image data and/or external data.

In some non-limiting embodiments or aspects, control system 102 may update at least one record (e.g., item record, object record, personnel record, patient record, procedure record, etc.) stored in at least one database (e.g., memory 104) based on the at least one status of the item, object, personnel, and/or patient. For example, a contamination status of an item may be updated by control system 102 by sending an update request to memory 104 to update a contamination attribute of an item record associated with the item. By way of further example, control system 102 may log (e.g., track based on time and other parameters) the use of items, location of items, statuses of items, involvement of personnel, uses of items by personnel, steps of a medical procedure, patient health information (e.g., blood pressure, heart rate, respiration, blood oxygen, etc.), and/or the like in one or more records stored in memory 104.

In some non-limiting embodiments or aspects, control system 102 may receive new image data from at least one imaging device 106 (e.g., on a recurring basis). Control system 102 may determine a suggested item at least partly based on the medical procedure and the new image data. For example, the suggested item may be an item that is used at a subsequent step (e.g., an immediately next or distantly subsequent step) in the medical procedure. By way of further example, if the medical procedure is an open-heart surgery for a patient, when the surgery is nearing completion, the suggested item may be a chest tube that is inserted to drain fluid or air from the chest cavity after surgery. Control system 102 may determine a suggested item based on items available in the room (e.g., using the above-described method of identifying items with machine-learning models) and/or a set of pre-approved items used for the medical procedure (e.g., a list or table of items that are appropriate to be used for the medical procedure). Control system 102 may then generate a notification via at least one computing device 105 (e.g., a medical computer cart, a workstation on wheels (WOW), a mobile device, etc.) positioned in the room. The notification may identify the suggested item (e.g., by name, sight, or sound) and may include one or more of: a textual description of the suggested item (e.g., a written word-based statement related to identity, use, location, and/or the like, such as “long needle holder for the 4-0 suture”, “gauze pack located on medical instrument stand”, etc.), an aural description of the suggested item (e.g., an audibilized message conveying identity, use, location, and/or the like, such as the generated sound of someone speaking “Metzenbaum scissors for dissection of soft tissue”, “Yankauer suction tip located in bedside tray”, etc.), a visual depiction of the suggested item (e.g., an image showing where in the room the item is, an image showing an example of what the item looks like, a video showing how to operate the item etc.), or any combination thereof.

In some non-limiting embodiments or aspects, control system 102 may determine a location of the suggested item in the room based on the new image data. For example, control system 102 may determine an absolute location (e.g., specific position, such as coordinates in the room) and/or a relative location (e.g., associated with a storage container, surface, person, etc.). The notification generated by control system 102 via at least one computing device 105 may identity the absolute and/or relative location of the suggested item. To illustrate, the notification may include a textual description, an aural description, and/or a visual depiction of absolute location and/or relative location of the item, such as a display of the sentence “the 7-inch vascular clamp for securing the axillary artery can be found at the top right of the medical instrument stand”, alongside a narration of the same and a depiction of the clamp.

In some non-limiting embodiments or aspects, control system 102 may be configured to determine a contamination status of at least one item based on the image data received from at least one imaging device 106. The contamination status may be indicative of the at least one item being contaminated. For example, control system 102 may use a machine-learning model to determine a proportion of the item that is covered by visual disparities associated with contamination, such as soiling, blood, etc. By way of further example, control system 102 may track the item in the room relative to other items, objects, personnel, and/or patients, and determine based on locations and movement whether the item has been touched, used, moved, dropped, and/or the like, which may be indicative of contamination. By way of further example, control system 102 may look up (e.g., retrieve from memory 104) an item record of the specific item and determine that the current date is beyond an expiry date of the item.

In some non-limiting embodiments or aspects, control system 102 may update a patient record, including multiple times during the course of a medical procedure. Control system 102 may regularly repeat a series of analytic and recording-based steps to maintain a log of events during the medical procedure. For example, control system 102 may repeatedly receive image data from at least one imaging device 106, determine a status of the at least one item, and update the patient record based on the status of the at least one item. To illustrate, in a given time period, control system 102 may detect an item's use status via presence, movement, and/or associated personnel, such as the use of a scalpel for an incision on the patient. Control system 102 may, in response, update the patient record to record a “Time of Incision” attribute for the medical procedure. In this manner, the patient's record may accurately reflect what items were used on the patient, what time items were used, what items were placed in the patient, what medical procedure steps were completed, which personnel used certain items in the medical procedure, which personnel completed certain medical procedure steps, what time medical procedure steps were completed, and/or the like. Such techniques greatly improve the accuracy and depth of a patient record surrounding a medical procedure.

Further to that end, control system 102 may be configured to, based on image data from at least one imaging device 106, determine at least one personnel in the room. For example, control system 102 may input image data to a machine-learning model trained on images of known medical personnel associated with the room (e.g., such as staff pictures for the hospital), to identify personnel who are present for the medical procedure. When updating the one or more records stored in memory 104, control system 102 may associate one or more personnel with the at least one item in the records. This may allow more precise auditing of item records, to determine where items are stored, which personnel took possession of certain items, which items need to be cleaned, which personnel mislaid an item, and/or the like. Moreover, control system 102 may be configured to determine a use of the at least one item based on the image data. The use of the item (e.g., a binary state such as “used” or “unused”, a description of the use, a value of the use, etc.) may be associated with at least one step of a plurality of steps of the medical procedure. When updating the one or more records stored in memory 104, control system 102 may log (e.g., record relative to time) the use of the at least one item based on the medical procedure (e.g., an association with a step of the medical procedure) and a time when the use was completed (e.g., time when initiated, time period of use, time period of removal, etc.).

In some non-limiting embodiments or aspects, control system 102 may be configured to filter image data related to humans (e.g., personnel, patients, etc.) to protect identity, modesty, confidentiality, and/or the like. As such, control system 102 may be configured to filter the image data in real-time to censor at least one body part of a personnel, a patient, and/or the like. For example, one or more faces of personnel and/or patients may be censored (e.g., blurred, redacted, omitted, masked, etc.) from the image data that may be later processed, analyzed, stored, displayed, and/or the like, by control system 102. By way of further example, sensitive body parts (e.g., genitals, sex organs, internal cavities, etc.) may be censored from the image data that may be later processed, analyzed, stored, displayed, and/or the like, by control system 102. In some non-limiting embodiments or aspects, control system 102 may temporarily store the image data in a memory buffer and apply an isolated (e.g., closed-system) machine-learning model that is trained to identify the body part(s) to be censored. Control system 102 may then censor the portions of the image data corresponding to what the isolated machine-learning model classifies as censorable. Control system 102 may then release the censored image data from the memory buffer for further processing, analysis, storage, display, and/or the like. Additionally, or alternatively, control system 102 may purge (e.g., delete) uncensored image data from the memory buffer that contains censorable portions, so that censorable content is not used for later processing, analysis, storage, display, and/or the like.

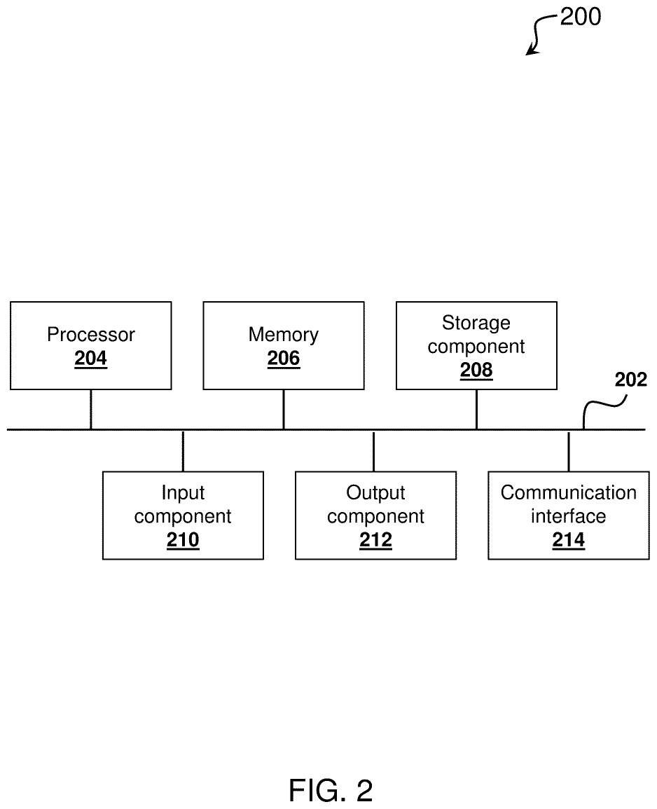

Referring now to FIG. 2, shown is a diagram of example components of a device 200, according to non-limiting embodiments. Device 200 may correspond to one or more devices of control system 102, memory 104, computing device 105, imaging device 106, and/or communication network 108, as an example. In some non-limiting embodiments, such systems or devices may include at least one device 200 and/or at least one component of device 200. The number and arrangement of components shown are provided as an example. In some non-limiting embodiments, device 200 may include additional components, fewer components, different components, or differently arranged components than those shown. Additionally, or alternatively, a set of components (e.g., one or more components) of device 200 may perform one or more functions described as being performed by another set of components of device 200.

As shown in FIG. 2, device 200 may include bus 202, processor 204, memory 206, storage component 208, input component 210, output component 212, and communication interface 214. Bus 202 may include a component that permits communication among the components of device 200. In some non-limiting embodiments or aspects, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read only memory (ROM), and/or another type of dynamic or static storage device (e.g., flash memory, magnetic memory, optical memory, etc.) that stores information and/or instructions for use by processor 204.

With continued reference to FIG. 2, storage component 208 may store information and/or software related to the operation and use of device 200. For example, storage component 208 may include a hard disk (e.g., a magnetic disk, an optical disk, a magneto-optic disk, a solid-state disk, etc.) and/or another type of computer-readable medium. Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.). Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium may include any non-transitory memory device. A memory device includes memory space located inside of a single physical storage device or memory space spread across multiple physical storage devices. Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to an arrangement of software, device(s), and/or hardware for performing and/or enabling one or more functions (e.g., actions, processes, steps of a process, and/or the like). For example, “a processor configured to” may refer to a processor that executes software instructions (e.g., program code) that cause the processor to perform one or more functions.

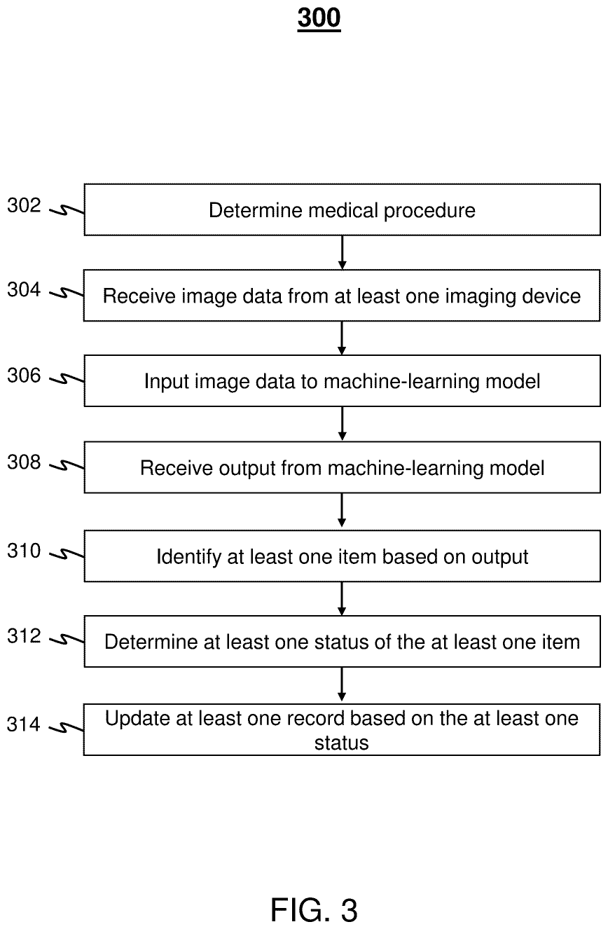

Referring now to FIG. 3, shown is a flow diagram for a method 300 for medical procedure-based inventory management, according to some non-limiting embodiments or aspects. The steps shown in FIG. 3 are for example purposes only. It will be appreciated that additional, fewer, different, and/or a different order of steps may be used in non-limiting embodiments or aspects. In some non-limiting embodiments or aspects, a step may be automatically performed in response to performance and/or completion of a prior step. In some non-limiting embodiments or aspects, one or more of the steps of method 300 may be performed (e.g., completely, partially, and/or the like) by control system 102. In some non-limiting embodiments or aspects, one or more of the steps of method 300 may be performed (e.g., completely, partially, and/or the like) by another system, another device, another group of systems, or another group of devices, separate from or including control system 102.

As shown in FIG. 3, at step 302, method 300 may include determining a medical procedure. For example, control system 102 may determine a medical procedure to be performed on a patient.

As shown in FIG. 3, at step 304, method 300 may include receiving image data from at least one imaging device. For example, control system 102 may receive image data from at least one imaging device positioned in a room in which the medical procedure is performed.

As shown in FIG. 3, at step 306, method 300 may include inputting image data to a machine-learning model. For example, control system 102 may input the image data to at least one machine-learning model trained on images of a plurality of items.

As shown in FIG. 3, at step 308, method 300 may include receiving output from the machine-learning model. For example, control system 102 may receive an output from the at least one machine-learning model associated with a classification of the image data.

As shown in FIG. 3, at step 310, method 300 may include identifying at least one item based on the output. For example, control system 102 may identify at least one item based on the output from the at least one machine-learning model.

As shown in FIG. 3, at step 312, method 300 may include determining at least one status of the at least one item. For example, control system 102 may determine at least one status of the at least one item based at least partly on at least one of: a location of the at least one item in the room, a movement of the at least one item in the room, a time, or any combination thereof.

As shown in FIG. 3, at step 314, method 300 may include updating at least one record based on the at least one status. For example, control system 102 may update at least one record stored in at least one database based on the at least one status of the at least one item.

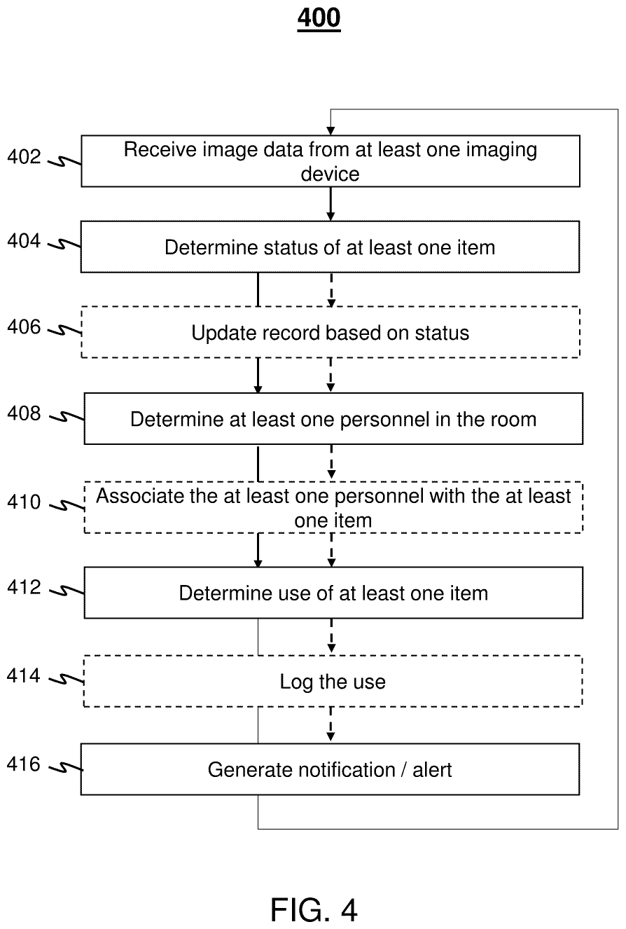

Referring now to FIG. 4, shown is a flow diagram for a method 400 for medical procedure-based inventory management, according to some non-limiting embodiments or aspects. The steps shown in FIG. 4 are for example purposes only. It will be appreciated that additional, fewer, different, and/or a different order of steps may be used in non-limiting embodiments or aspects. In some non-limiting embodiments or aspects, a step may be automatically performed in response to performance and/or completion of a prior step. In some non-limiting embodiments or aspects, one or more of the steps of method 400 may be performed (e.g., completely, partially, and/or the like) by control system 102. In some non-limiting embodiments or aspects, one or more of the steps of method 400 may be performed (e.g., completely, partially, and/or the like) by another system, another device, another group of systems, or another group of devices, separate from or including control system 102. As shown, one or more steps of method 400 may be repeatedly performed (e.g., looped), until an end condition is met (e.g., the medical procedure is completed, a user stops the loop, a time period has elapsed, etc.).

As shown in FIG. 4, at step 402, method 400 may include receiving image data from at least one imaging device. For example, control system 102 may receive image data from at least one imaging device (e.g., a camera).

As shown in FIG. 4, at step 404, method 400 may include determining a status of at least one item. For example, control system 102 may determine at least one status of the at least one item.

As shown in FIG. 4, at step 406, method 400 may include updating a record based on the status. For example, control system 102 may update at least one record (e.g., a patient record) based on at least one status of the at least one item.

As shown in FIG. 4, at step 408, method 400 may include determining at least one personnel in the room. For example, control system 102 may update at least one personnel in the room based on the image data.

As shown in FIG. 4, at step 410, method 400 may include associating the at least one personnel with the at least one item. For example, control system 102, when updating the at least one record, may associated the at least one personnel with the at least one item in the at least one record.

As shown in FIG. 4, at step 412, method 400 may include determining a use of the at least one item. For example, control system 102 may determine a use of the at least one item based on the image data. The use may be associated with at least one step of a plurality of steps of the medical procedure.

As shown in FIG. 4, at step 414, method 400 may include logging the use. For example, control system 102 may, when updating the at least one record, log the use of the at least one item based on the medical procedure and a time when the use was completed.

As shown in FIG. 4, at step 416, method 400 may include generating a notification and/or alert. For example, control system 102 may generate an alert via at least one computing device positioned in the room (e.g., computing device 105). The alert may be associated with at least one status (e.g., indicative of an item being contaminated), a set of pre-approved items used for the medical procedure (e.g., indicative of an incorrect item being used), and/or the like. Additionally, or alternatively, control system 102 may generate a notification via at least one computing device (e.g., computing device 105) positioned in the room. The notification may identify an item (e.g., a suggested item) and include at least one of a textual description of an item, an aural description of an item, a visual description of an item, or any combination thereof.

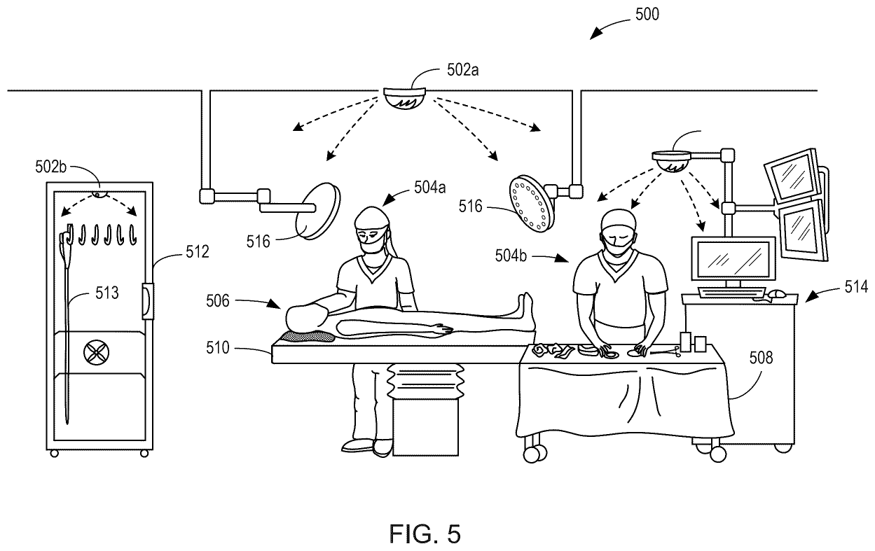

Referring now to FIG. 5, FIG. 5 is an illustration of a procedure room 500 configured for a method for medical procedure-based inventory management, according to some non-limiting embodiments or aspects. The depicted procedure room 500 is an operation room that is currently configured for a medical procedure for a patient 506, which may include an endoscopy. Two personnel are depicted as present in room 500, including a doctor 504a and a nurse 504b. Positioned in room 500 are a plurality of imaging devices, including a first camera 502a, a second camera 502b, and a third camera 502c. First camera 502a is mounted on a ceiling of room 500 and has a observable range that may cover a majority of room 500, including operation lights 516, surgical bed 510, medical instrument stand 508, medical computer cart 514 (e.g., including one or more computing devices with displays, speakers, input devices, and/or the like), storage cabinet 512, doctor 504a, nurse 504b, and patient 506. Second camera 502b is mounted inside storage cabinet 512 and has a localized observable range inside storage cabinet 512, including view of any items stored therein, including endoscope 513. Third camera 502c is mounted on medical computer cart 514 and has a localized observable range that includes medical computer cart 514, medical instrument stand 508, nurse 504b, and potentially portions of surgical bed 510 and patient 506. Cameras 502a, 502b, 502c may be configured to capture image data in a stream of images and communicate those images to control system 102.

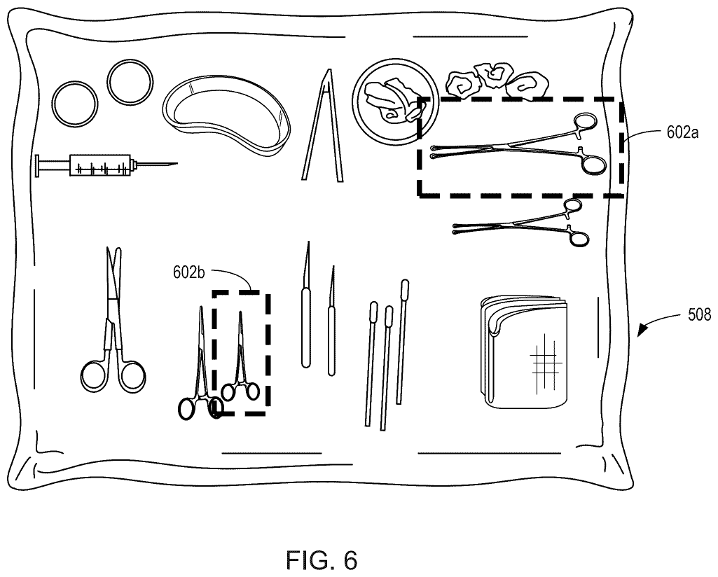

During the medical procedure, control system 102 may be positioned in room 500 (e.g., associated with a computing device of medical computer cart 514) and/or external to room 500, such as in a server cluster (e.g., located in a hospital server room, a cloud storage system external to hospital, etc.). Control system 102 may receive image data from third camera 502c and identify one or more items stored on medical instrument stand 508. As doctor 504a and/or nurse 504b interact with items stored on medical instrument stand 508, control system 102 may track, classify, and/or identify those items and determine ongoing statuses of those items, including whether those items have been used, contaminated, cleaned, and/or the like. For further detailed depiction of the image data analysis from third camera 502c, refer to FIG. 6.

Similarly, control system 102 may receive image data from first camera 502a and identify one or more items located in room 500. As doctor 504a and/or nurse 504b interact with items in room 500, control system 102 may track, classify, and/or identify those items and determine ongoing statuses of those items, including whether those items have been used, contaminated, cleaned, and/or the like. Moreover, control system 102 may receive image data from second camera 502b and identify one or more items stored in storage cabinet 512 (e.g., endoscope 513). As doctor 504a and/or nurse 504b interact with items in storage cabinet 512, control system 102 may track, classify, and/or identify those items and determine ongoing statuses of those items, including whether those items have been used, contaminated, cleaned, and/or the like.

Control system 102 may reference image data from one or more cameras (e.g., first camera 502a, second camera 502b, third camera 502c) to determine statuses of items. During the medical procedure, control system 102 may determine a suggested item at least partly based on the medical procedure and the image data received from first camera 502a, second camera 502b, and/or third camera 502c. Control system 102 may generate a notification via medical computer cart 514 to identify the suggested item using a textual description, an aural description, a visual depiction, and/or the like. For example, control system 102 may determine that patient 506 has been prepped to begin the endoscopy, and for that purpose, control system 102 may identify endoscope 513, such as by displaying text on a screen that says “Retrieve endoscope from storage cabinet”, audibilizing the phrase “Retrieve an endoscope from the storage cabinet”, and/or displaying a picture of endoscope 513 (e.g., using captured image data from room 500, stored image data in memory 104 of the same or similar items, and/or the like).

Control system 102 may determine a location of the suggested item, e.g., endoscope 513, based on the image data received from second camera 502b that shows endoscope 513 hanging on a hook in storage cabinet 512. Using the determined location, the location of the endoscope 513 may be included in the notification to doctor 504a and/or nurse 504b, such as to specify which storage container the item is located in, where the storage container is located, which partition (e.g., hook) of the storage container the item is located in, etc. Doctor 504a and/or nurse 504b may proceed to retrieve the suggested item from the determined location, based on referencing the notification generated by control system 102.

As control system 102 monitors medical procedure using image data from cameras 502a, 502b, 502c, control system 102 may detect the use of endoscope 513 when endoscope 513 is removed from storage cabinet 512 and brought over to surgical bed 510. The removal of endoscope 513 from storage cabinet 512 by doctor 504a may be logged in an item record for endoscope 513, a patient record for patient 506, and/or a procedure record for the endoscopy, that is stored in memory 104 (e.g., in a data storage device inside or outside room 500). In this manner, the status of the item may be updated accurately to reflect time of use/movement, personnel associated with the use/movement, and/or the like, promoting enhanced inventory management and record keeping. An identity of doctor 504a (e.g., personnel identifier) may be associated with a record of endoscope 513 by control system 102 when doctor 504a collects endoscope 513 from storage cabinet 512.

Control system 102 may be further configured to generate an alert via at least one computing device 105 (e.g., medical computer cart 514, a mobile device associated with doctor 504a or nurse 504b, etc.) positioned in room 500. The alert may be associated with a status indicative of an item being contaminated (e.g., soiled, infected, exposed to bacteria, expired, etc.). For example, as nurse 504b is organizing medical instrument stand 508, third camera 502c may communicate image data to control system 102 related to items stored on medical instrument stand 508. In some non-limiting embodiments or aspects, control system 102 may determine that an item on medical instrument stand 508 is contaminated (e.g., from visual differences detectable on the surface of the item, from expiry dates associated with the item, from movement of the item to a non-clean surface, from association with one or more personnel and/or patients, etc.). In some non-limiting embodiments or aspects, control system 102 may determine that an item on medical instrument stand 508 is not associated with a set of pre-approved items used for the medical procedure. For example, control system 102 may reference a database of information previously configured by personnel related to one or more items that are approved for use with the medical procedure and compare detected items on the medical instrument stand 508 with information stored in the database (e.g., memory 104). An item that is not associated with an approved item in the database for the medical procedure may be determined to be unapproved. In response to determining that an item is contaminated, unapproved, and/or the like, control system 102 may generate an alert (e.g., a text alert, an audible alert, an image alert, a video alert, a haptic alert, etc.) to doctor 504a and/or nurse 504b through at least one computing device 105 (e.g., medical computer cart 514, a mobile device associated with doctor 504a or nurse 504b, etc.). The alert may convey information about the problematic item, including information identifying the item, its problematic status (e.g., contaminated, unapproved, etc.), and/or a suggested item to replace the problematic item.

Control system 102 may continually (e.g., regularly repeat) during the medical procedure perform a series of steps to update a record (e.g., the patient record for patient 506). For example, control system 102 may regularly receive image data from first camera 502a, second camera 502b, and/or third camera 502c and process the image data, partly using one or more machine-learning models, to identify items, track the location of items, track the movement of items, associate items with personnel, associate items with patient 506, determine use of the item for the medical procedure, detect contamination, detect unapproved items, and/or the like. In response to control system 102 determining at least one status of at least one item in room 500, control system 102 may update an item record, patient record, procedure record, and/or the like based on the at least one status of the at least one item. To illustrate, control system 102 may detect, based on image data from second camera 502b and/or first camera 502a, that doctor 504a collected endoscope 513 from storage cabinet 512 and began the endoscopy procedure at a particular time. Control system 102 may then update the patient record to reflect the time that the endoscopy began and may associate one or more items (e.g., endoscope 513) and/or personnel (e.g., doctor 504a) with the beginning of the endoscopy. To further illustrate, control system 102 may subsequently detect, based on image data from first camera 502a and/or third camera 502c, that doctor 504a handed nurse 504b endoscope 513 after use and nurse 504b moved endoscope 513 to a post-procedure processing station (e.g., for subsequent cleaning). Control system 102 may then update the patient record to reflect the time that the endoscopy concluded and may update an item record for endoscope 513 to reflect that endoscope 513 is contaminated, that endoscope 513 was last in the custody of nurse 504b, and that the current location of endoscope 513 is in the post-procedure processing station. It will be appreciated that recurring image data communicated to control system 102 may be continually used to identify items, item statuses, and update records (e.g., patient records, item records, procedure records, personnel records, etc.).

Control system 102 may further be configured to use a machine-learning model trained to identify personnel and/or patients 506. For example, the machine-learning model may be trained to identify individuals based on badges, faces, partially covered faces, eyes, height, skin color, stature, gait, color of clothing, and/or other identifying indicia (e.g., as may be provided with consent by traceable individuals and under appropriate security protocols surrounding the storage and transfer of personal information). Additionally, or alternatively, control system 102 may receive data identifying personnel based on self-identifying mechanisms, such as RFID tags worn by personnel, barcodes on badges, manual input by personnel identifying themselves to control system 102, and/or the like. Control system 102 may use the identification of personnel, such as doctor 504a and/or nurse 504b, to associate personnel identifiers (e.g., names, identification numbers, identification codes, etc.) with detected items, patients 506, and/or the like. Control system 102 may use the identification of patients 506 to associate patient identifiers (e.g., names, identification numbers, identification codes, etc.) with detected items, personnel, and/or the like.

Control system 102 may be further configured to censor at least one body part of personnel (e.g., doctor 504a, nurse 504b) or of patient 506. For example, control system 102 may, before downstream processing, securely load image data received from first camera 502a, second camera 502b, and/or third camera 502c into a screening pipeline (e.g., a buffer). Control system 102 may apply one or more machine-learning models to identify censorable body parts, including, but not limited to, faces, exposed body cavities, sex organs, and/or the like. Control system 102 may then filter the image data in real-time to censor the at least one body part of the patient. For example, control system 102 may apply a mask to or obscure (e.g., blur) the portion of the image data that is associated with the censored body part. By way of another example, control system 102 may remove (e.g., scrub, delete) the portion of the image data that is associated with the censored body part. For example, the portion of the image data associated with the face of patient 506 may be blurred (e.g., pixelated) to protect the identity of patient 506. Any image data that is stored in memory 104 and/or released from the real-time screening pipeline that is associated with individuals (e.g., personnel, patient 506, etc.) may be stored in an encrypted, anonymized, and/or de-identified format consistent with predetermined security protocols.

Referring now to FIG. 6, FIG. 6 is an illustration of a top surface of a medical instrument stand 508 for use with a method for medical procedure-based inventory management, according to some non-limiting embodiments or aspects. The depicted view of FIG. 6 includes dashed lines indicating views of items, of the many items stored on medical instrument stand 508, that may be classified by control system 102 according to the methods described herein.

In some non-limiting embodiments or aspects, control system 102 may receive image data from at least one imaging device positioned in a room in which the medical procedure is performed. For example, the view of medical instrument stand 508, as shown in FIG. 6, may be captured by third camera 502c (shown in FIG. 5). Third camera 502c may communicate image data of medical instrument stand 508 to control system 102 for processing. The image data transmitted from third camera 502c to control system 102 may include all of the image data collected by third camera 502c at a given time step, or a portion of all the image data collected by third camera 502c at the given time step. Control system 102 may receive the image data transmitted from third camera 502c and input the image data (e.g., all of the image data received, a portion of the image data received, etc.) to at least one machine-learning model trained on images of a plurality of items. For example, control system 102 may input all of the image data represented by FIG. 6 into a machine-learning model. Additionally, or alternatively, control system 102 may input a portion of image data, corresponding to first view 602a or second view 602b, to a machine-learning model to determine a classification for an item contained in first view 602a or second view 602b. It will be appreciated that while only two exemplary views (e.g., first view 602a, second view 602b) are provided for illustration, any number of views may be collected and analyzed in the image data for classification by control system 102.

In some non-limiting embodiments or aspects, control system 102 may receive an output from the at least one machine-learning model associated with a classification of the image data. For example, if control system 102 input all of the image data of FIG. 6 into the machine-learning model, the machine-learning model may generate an output associated with one or more classifications of items contained therein, including a classification of the item in first view 602a (e.g., sponge forceps), a classification of the item in second view 602b (e.g., hemostatic forceps), and/or the like. By way of another example, if control system 102 input a portion of the image data associated with first view 602a into the machine-learning model, the machine-learning model may generate an output associated a classification of the item contained within first view 602a (e.g., sponge forceps). In response to receiving the output from the at least one machine-learning model, control system 102 may proceed to identify one or more items based on the output(s), determine one or more statuses for each item, and update one or more records associated with the identified items and/or statuses (e.g., item records, patient records, procedure records, etc.).

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A system comprising:
at least one processor configured to:
determine a medical procedure to be performed on a patient;
receive image data from at least one imaging device positioned in a room in which the medical procedure is performed;
input the image data to at least one machine-learning model trained on images of a plurality of items;
receive an output from the at least one machine-learning model associated with a classification of the image data;
identify at least one item based on the output;
determine at least one status of the at least one item based at least partly on at least one of: a location of the at least one item in the room, a movement of the at least one item in the room, a time, or any combination thereof; and
update at least one record stored in at least one database based on the at least one status of the at least one item.

2. The system of claim 1, wherein the at least one processor is also configured to:
receive new image data from the at least one imaging device;
determine a suggested item at least partly based on the medical procedure and the new image data; and
generate a notification via at least one computing device positioned in the room, the notification identifying the suggested item and comprising at least one of: a textual description of the suggested item, an aural description of the suggested item, a visual depiction of the suggested item, or any combination thereof.

3. The system of claim 2, wherein the at least one processor is also configured to determine a location of the suggested item in the room based on the new image data, wherein the notification generated via the at least one computing device further identifies the location of the suggested item.

4. The system of claim 1, wherein the at least one processor is also configured to generate an alert via at least one computing device positioned in the room, the alert associated with at least one of the following: the at least one status comprising a status indicative of the at least one item being contaminated, the at least one item not being associated with a set of pre-approved items used for the medical procedure, or any combination thereof.

5. The system of claim 4, wherein the at least one processor is also configured to determine a contamination status of the at least one status based on the image data, the contamination status indicative of the at least one item being contaminated.

6. The system of claim 1, wherein the at least one record comprises a patient record, and wherein the at least one processor is also configured to regularly repeat, during the medical procedure:
receiving the image data from the at least one imaging device;
determining the at least one status of the at least one item; and
updating the patient record based on the at least one status of the at least one item.

7. The system of claim 1, wherein the at least one processor is also configured to:
receive new image data from the at least one imaging device; and
determine at least one personnel in the room based on the new image data; and
wherein, when updating the at least one record, the at least one processor is configured to:
associate the at least one personnel with the at least one item in the at least one record.

8. The system of claim 7, wherein the at least one processor is further configured to determine a use of the at least one item based on the image data, the use of the at least one item associated with at least one step of a plurality of steps of the medical procedure; and
wherein, when updating the at least one record, the at least one processor is configured to:
log the use of the at least one item based on the medical procedure and a time when the use was completed.

9. The system of claim 1, wherein the at least one processor is also configured to filter the image data in real-time to censor at least one body part of the patient.

10. The system of claim 1, wherein the at least one processor is configured to determine at least one of: the location of the at least one item in the room, the movement of the at least one item in the room, the time, or any combination thereof.

11. A computer-implemented method comprising:
determining, with at least one processor, a medical procedure to be performed on a patient;
receiving, with at least one processor, image data from at least one imaging device positioned in a room in which the medical procedure is performed;
inputting, with at least one processor, the image data to at least one machine-learning model trained on images of a plurality of items;
receiving, with at least one processor, an output from the at least one machine-learning model associated with a classification of the image data;
identifying, with at least one processor, at least one item based on the output;
determining, with at least one processor, at least one status of the at least one item based at least partly on at least one of: a location of the at least one item in the room, a movement of the at least one item in the room, a time, or any combination thereof; and
updating, with at least one processor, at least one record stored in at least one database based on the at least one status of the at least one item.

12. The computer-implemented method of claim 11, further comprising:
receiving, with at least one processor, new image data from the at least one imaging device;
determining, with at least one processor, a suggested item at least partly based on the medical procedure and the new image data; and
generating, with at least one processor, a notification via at least one computing device positioned in the room, the notification identifying the suggested item and comprising at least one of: a textual description of the suggested item, an aural description of the suggested item, a visual depiction of the suggested item, or any combination thereof.

13. The computer-implemented method of claim 11, further comprising generating, with at least one processor, an alert via at least one computing device positioned in the room, the alert associated with at least one of the following: the at least one status comprising a status indicative of the at least one item being contaminated, the at least one item not being associated with a set of pre-approved items used for the medical procedure, or any combination thereof.

14. The computer-implemented method of claim 11, wherein the at least one record comprises a patient record, the method further comprising regularly repeating, during the medical procedure:
receiving, with at least one processor, the image data from the at least one imaging device;
determining, with at least one processor, the at least one status of the at least one item; and
updating, with at least one processor, the patient record based on the at least one status of the at least one item.

15. The computer-implemented method of claim 11, further comprising:
receiving, with at least one processor, new image data from the at least one imaging device; and
determining, with at least one processor, at least one personnel in the room based on the new image data;
wherein updating the at least one record further comprises:
associating the at least one personnel with the at least one item in the at least one record.

16. A computer program product comprising at least one non-transitory computer-readable medium comprising program instructions that, when executed by at least one processor, cause the at least one processor to:
determine a medical procedure to be performed on a patient;
receive image data from at least one imaging device positioned in a room in which the medical procedure is performed;
input the image data to at least one machine-learning model trained on images of a plurality of items;
receive an output from the at least one machine-learning model associated with a classification of the image data;
identify at least one item based on the output;
determine at least one status of the at least one item based at least partly on at least one of: a location of the at least one item in the room, a movement of the at least one item in the room, a time, or any combination thereof; and
update at least one record stored in at least one database based on the at least one status of the at least one item.

17. The computer program product of claim 16, wherein the program instructions also cause the at least one processor to:
receive new image data from the at least one imaging device;
determine a suggested item at least partly based on the medical procedure and the new image data; and
generate a notification via at least one computing device positioned in the room, the notification identifying the suggested item and comprising at least one of: a textual description of the suggested item, an aural description of the suggested item, a visual depiction of the suggested item, or any combination thereof.

18. The computer program product of claim 16, wherein the program instructions also cause the at least one processor to generate an alert via at least one computing device positioned in the room, the alert associated with at least one of the following: the at least one status comprising a status indicative of the at least one item being contaminated, the at least one item not being associated with a set of pre-approved items used for the medical procedure, or any combination thereof.

19. The computer program product of claim 16, wherein the at least one record comprises a patient record, and wherein the program instructions also cause the at least one processor to regularly repeat, during the medical procedure:
receiving the image data from the at least one imaging device;
determining the at least one status of the at least one item; and
updating the patient record based on the at least one status of the at least one item.

20. The computer program product of claim 16, wherein the program instructions also cause the at least one processor to:
receive new image data from the at least one imaging device; and
determine at least one personnel in the room based on the new image data; and
wherein the program instructions that cause the at least one processor to update the at least one record cause the at least one processor to:
associate the at least one personnel with the at least one item in the at least one record.
